(19) United States
(12) Patent Application Publication
(10) Pub. No.: US 2020/0377833 A1
(43) Pub. Date: Dec. 3, 2020

(54) CELL PICKING DEVICE

(71) Applicant: SHIMADZU CORPORATION, Kyoto (JP)

(72) Inventors: Takashi INOUE, Kyoto (JP); Kenji TAKAKURA, Kyoto (JP); Yoshitaka NODA, Kyoto (JP); Yasuaki MATSUNAGA, Kyoto (JP); Mika OKADA, Kyoto (JP); Akari TAKEDA, Kyoto (JP)

(21) Appl. No.: 16/969,859

(22) PCT Filed: Mar. 16, 2018
(86) PCT No.: PCT/JP2018/010488

§ 371 Date: Aug. 13, 2020

(51) Int. Cl. C12M 1/26; B01L 9/00; C12M 1/36

(52) U.S. Cl. CPC C12M 1/265; G02B 21/0004; C12M 41/48; B01L 9/54

(57) ABSTRACT

A cell picking device includes a sucker having a pipette tip attached to a tip portion of the sucker and is used to suck the cell from a tip of the pipette tip, a driver configured to cause the sucker to perform a suction operation and move the sucker in a horizontal-plane direction and an axial direction of the pipette tip with the sucker inclined with respect to a vertical direction. A controller moves the sucker such that the tip of the pipette tip moves in a horizontal direction while being in contact with a bottom surface of the container installed on the sample mounting stage in a predetermined position on the sample mounting stage to desorb a cell arranged in the predetermined position from the bottom surface of the container, and suck the cell desorbed from the bottom surface of the container from the tip of the pipette tip.

### TECHNICAL FIELD

The present invention relates to a cell picking device for picking a specific cell in a container using a microscope.

### BACKGROUND ART

In a case where picking a specific cell from a container such as a cell culture container, a worker sucks a subject cell generally and manually by using suction equipment such as a pipette while checking the position of the cell to be picked with a microscope. However, such work requires skill and cannot be done easily by anyone.

A device for assisting the above-mentioned cell picking work has been suggested (See Patent Document 1.) The suggested device is configured to three-dimensionally move a sucker having a pipette tip attached to its tip by a transporter, and adjust the position of the tip of the pipette tip to the position of the cell to be picked while optically detecting the tip of the pipette tip by a detector.

[Patent Document 1] JP 2016-112012 A

### SUMMARY OF INVENTION

### Technical Problem

As described in Patent Document 1, in the configuration for adjustment of the position of the tip of the pipette tip to the position of the cell to be picked, the smaller the size of the cell to be picked is, the higher the movement resolution of the pipette tip is required to be. Therefore, it requires time to move the pipette tip and adjust the position of the tip when picking a cell at a distance, and there is a problem that efficiency of the picking work is degraded.

Further, since a cell is adsorbed on the bottom surface of the container, it is difficult to suck the subject cell only by simply sucking the cell directly from above.

The present invention was conceived considering the above-mentioned problems, and an object of the present invention is to position a tip of a pipette tip highly efficiently with respect to a cell to be picked and suck the cell to be picked more reliably.

### Solution to Problem

A cell picking device according to the present invention is installed around a microscope and is used to pick a cell in a container installed on a sample mounting stage of the microscope. The cell picking device includes a sucker having a pipette tip attached to a tip portion of the sucker and is used to suck the cell from a tip of the pipette tip, a driver configured to cause the sucker to perform a suction operation and move the sucker in a horizontal-plane direction and an axial direction of the pipette tip with the sucker inclined with respect to a vertical direction, and a controller that controls the driver. The controller is configured to move the sucker such that the tip of the pipette tip moves in a horizontal direction while being in contact with a bottom surface of the container installed on the sample mounting stage in a predetermined position on the sample mounting stage to desorb a cell arranged in the predetermined position from the bottom surface of the container, and suck the cell desorbed from the bottom surface of the container from the tip of the pipette tip.

That is, in the cell picking device of the present invention, the position at which the tip of the pipette tip is to arrive during the cell picking is predetermined, and the cell to be picked is arranged at the predetermined position by a user with use of a microscope (the position in the field of view of the microscope). Therefore, the adjustment work of the position of the tip of the pipette tip during the cell picking is unnecessary. It is easy to arrange the cell to be picked in the field of view of the microscope. Then, in a case where the cell to be picked is arranged correctly at the predetermined position, the tip of the pipette tip is positioned quickly with respect to the cell to be picked. The sucker moves in the horizontal-plane direction and in the axial direction of the pipette tip while being inclined with respect to the vertical direction, so that the tip of the pipette tip can access the container on the sample mounting stage without interfering with a lens of the microscope. Further, the tip of the pipette tip moves in the horizontal direction while being in contact with the bottom surface of the container, and the cell arranged at the predetermined position is desorbed from the bottom surface of the container before being sucked. Thus, even in a case where the subject cell is adsorbed on the bottom surface of the container, the cell can be picked more reliably.

Furthermore, since a cell culture liquid stored in the cell culture container is costly, an amount of the cell culture liquid to be sucked together with the cell during the cell picking is preferably suppressed as much as possible. As such, in the present invention, the controller is preferably configured not to carry out suction by the sucker until the cell is desorbed from the bottom surface of the container, and suck the cell from the tip of the pipette tip after the cell is desorbed from the bottom surface of the container. Then, the amount of the cell culture liquid to be sucked from the tip of the pipette tip during the cell picking can be kept to a minimum.

The controller is preferably configured to move the tip of the pipette tip with the tip being in contact with the bottom surface of the container by obliquely pressing the tip of the pipette tip against the bottom surface of the container and elastically bending the tip of the pipette tip. Then, the cell adsorbed on the bottom surface of the container can be scraped off, and the cell to be picked can be sucked more reliably.

The driver may include a sucker angle adjustment mechanism for adjusting an inclination angle of the sucker with respect to the vertical direction. In a case where the inclination angle of the sucker is adjustable, the types of applicable microscopes and containers can be expanded, and the cell picking device can be highly versatile.

The sucker angle adjustment mechanism is preferably configured to rotate the sucker in a vertical plane by a motor. The sucker angle adjustment mechanism may be capable of manually adjusting the inclination angle of the sucker.

In the preferred embodiment of the cell picking device according to the present invention, a drive amount storage that stores information in regard to a drive amount of the driver required to position the tip of the pipette tip at the predetermined position is included, wherein the controller is configured to control the driver using the information stored in the drive amount storage when the cell is picked from the container installed on the sample mounting stage of the microscope.

In the above-mentioned case, the drive amount storage preferably stores information relating to the drive amount for each of a size of the pipette tip attached to the tip portion of the sucker, a type of the microscope and/or a type of the container installed on the sample mounting stage of the microscope, and the controller is preferably configured to determine a drive amount of the driver required to position the tip of the pipette tip at the predetermined position based on the information relating to the drive amount stored in the drive amount storage, and the size of the pipette tip, the type of the microscope and/or the type of the container that are set based on an input by a user, and control the driver using the determined drive amount. Then, the user only needs to select the size of the pipette tip, the type of microscope, and/or the type of container, and the device performs an appropriate cell picking operation based on the selection. Thus, the user can perform a setting work of the operating condition easily.

Further, although a disposable pipette tip is attached to the tip of the sucker, in a case where the pipette tip is detached manually, there is a risk that the users hand may become contaminated. In a case where the users hand is contaminated during the work, there is a problem that it is troublesome because the user is required to perform a disinfecting work of his or her hand each time.

As such, in the present invention, a tip detachment mechanism configured to detach the pipette tip from the sucker by utilizing an operation of moving the sucker in the axial direction of the pipette tip is preferably included. Then, the pipette tip can be automatically detached from the sucker, and the users hand can be prevented from being contaminated by a detachment work of the pipette tip. Since the tip detachment mechanism detaches the pipette tip by utilizing the operation of moving the sucker in the axial direction of the pipette tip, it is not necessary to provide a new driver for detaching the sucker, and an increase in cost can be suppressed.

### Advantageous Effects of Invention

In the cell picking device of the present invention, because the position at which the tip of the pipette tip is to arrive during the cell picking is predetermined, work efficiency of the cell picking is improved as compared to the case where the adjustment work of the position of the tip of the pipette tip is performed during the cell picking. Further, since moving while being inclined with respect to the vertical direction, the sucker can access the container on the sample mounting stage without interfering with a lens of the microscope. Furthermore, during the cell picking, the tip of the pipette tip moves in the horizontal direction while being in contact with the bottom surface of the container, and the cell adsorbed on the bottom surface of the container is desorbed before being sucked. Thus, the cell to be picked can be sucked reliably.

### BRIEF DESCRIPTION OF THE DRAWINGS

FIG. 1 is a perspective view schematically showing one inventive example of a cell picking device.

FIG. 2 is a block diagram schematically showing a control system of the same inventive example.

FIG. 3 is a conceptual diagram when a cell is picked from a container installed in a microscope with use of the cell picking device of the same inventive example.

FIG. 4 is a flow chart showing one example of a cell picking work implemented by the cell picking device of the same inventive example.

FIG. 5 is a conceptual diagram showing the process of arranging a tip of a pipette tip at a predetermined position in cell picking performed by the cell picking device of the same inventive example.

FIG. 6 is a conceptual diagram showing the process that continues after the process of FIG. 5 in the cell picking performed by the cell picking device of the same inventive example, and showing the process of desorbing the cell from the bottom surface of the container by the tip of the pipette tip.

FIG. 7 is a conceptual diagram showing the process that continues after the process of FIG. 6 in the cell picking performed by the cell picking device of the same inventive example, and showing the process of sucking a cell to be picked from the tip of the pipette tip.

FIG. 8 is a side view of a sucker drive mechanism showing an example of the structure of a sucker drive mechanism.

### DESCRIPTION OF EMBODIMENTS

One inventive example of a cell picking device according to the present invention will be described below with reference to the drawings.

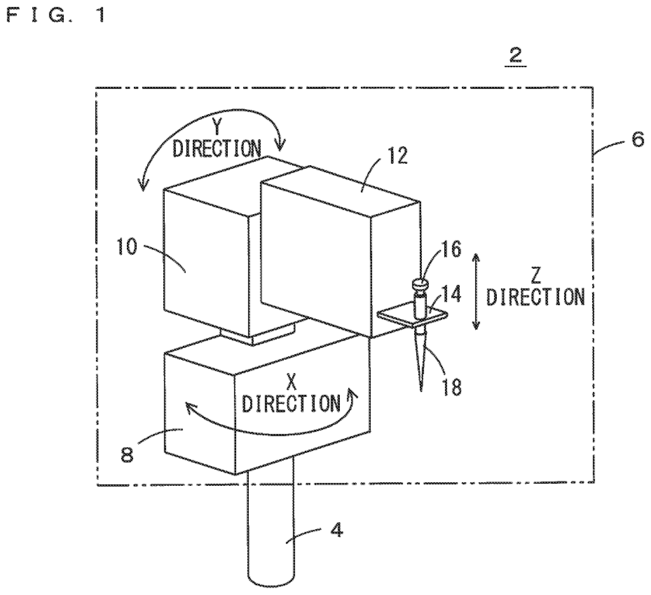

As shown in FIG. 1, the cell picking device 2 of this inventive example includes a driver 6 that holds and moves a pipette-type sucker 16 having a pipette tip 18 attached to its tip. The driver 6 includes a horizontal rotator 8 that is pivotally supported by a vertical shaft 4 and rotates in a horizontal plane, a vertical rotator 10 that is pivotally supported by the shaft which is attached to the horizontal rotator 8 and rotates in a vertical plane and a sucker drive mechanism 12 attached to the vertical rotator 10.

The sucker drive mechanism 12 includes a mechanism (not shown) for driving a suction operation by the sucker 16 in addition to a holding arm 14 for holding the sucker 16. The sucker drive mechanism 12 is configured to move the holding arm 14 in an axial direction of the pipette tip 18.

In this inventive example, the operating direction of the horizontal rotator 8, that is, the rotating direction in the horizontal plane is defined as an X direction, the operating direction of the vertical rotator 10, that is, the rotating direction in the vertical plane is defined as a Y direction, and the operating direction of the holding arm 14, that is, the axial direction of the pipette tip 18 is defined as a Z direction.

The driver 6 can move the sucker 16 in the three directions of the X, Y and Z directions by having the above-mentioned configuration. It is possible to move the sucker 16 in the horizontal-plane direction by rotating the horizontal rotator 8. It is possible to change an inclination angle of the sucker 16 with respect to a vertical axis direction by rotating the vertical rotator 10. Further, it is possible to adjust the height of the position of the tip of the pipette tip 18 by moving the holding arm 14 in the axial direction of the pipette tip 18. The vertical rotator 10 implements a sucker angle adjustment mechanism for adjusting the inclination angle of the sucker 16 with respect to the vertical axis direction.

While the horizontal rotator 8 is configured to move the sucker 16 in the horizontal-plane direction by rotating in the horizontal-plane direction in this inventive example, the present invention is not limited to this. A mechanism for moving the sucker 16 in two directions that are orthogonal to each other in the horizontal plane may be used.

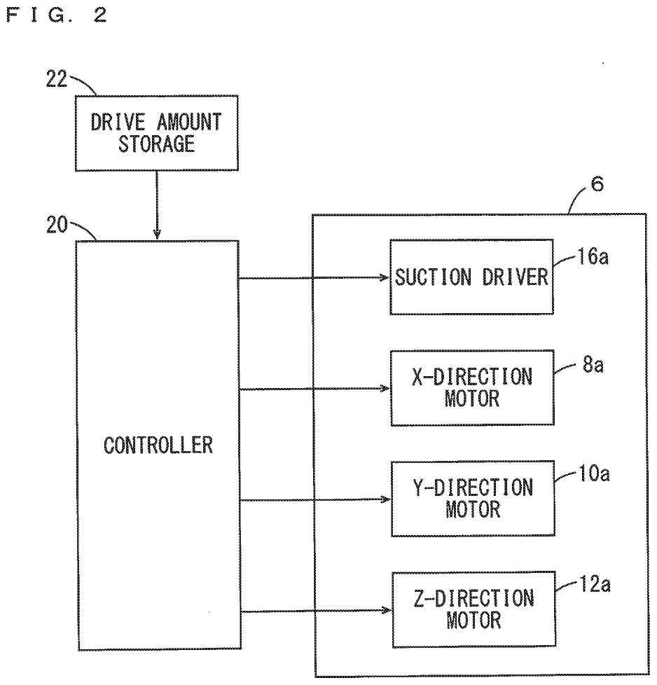

FIG. 2 is a block diagram showing one example of a control system of the cell picking device 2.

The driver 6 includes an X-direction motor 8a for rotating the horizontal rotator 8 in the horizontal plane, a Y-direction motor 10a for rotating the vertical rotator 10 in the vertical plane, a Z-direction motor 12a for moving the holding arm 14 in the axial direction of the pipette tip 18 and a suction driver 16a for driving a suction operation of the sucker 16. A controller 20 controls a drive amount and drive timing of these components. The controller 20 is configured to control the operations of the X-direction motor 8a, the Y-direction motor 10a, the Z-direction motor 12a and the suction driver 16a based on the information in regard to the drive amount and the drive timing stored in a drive amount storage 22.

The controller 20 is a function implemented when a computing element such as a microcomputer executes a program, and the drive amount storage 22 is a function implemented by part of a storage region provided in a storage device.

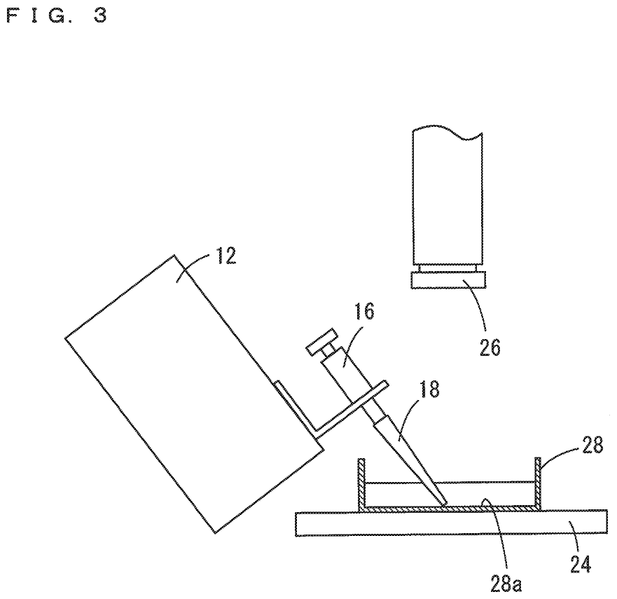

This cell picking device 2 is attached to a general-purpose microscope and can pick a specific cell in a container 28 installed on a sample mounting stage 24 of the microscope as shown in FIG. 3. The drive amount storage 22 of the cell picking device 2 stores the drive amounts of the X-direction motor 8a, the Y-direction motor 10a and the Z-direction motor 12a required to move the tip of the pipette tip 18 attached to the sucker 16 from an initial position to a “predetermined position” on the sample mounting stage 24 of the microscope. The “predetermined position” at which the tip of the pipette tip 18 is to arrive is a position where a cell arranged in a position in a field of view of the microscope can be scraped off as shown in FIG. 5.

Further, the drive amount storage 22 is preferably configured to store the information in regard to the drive amounts of the X-direction motor 8a, the Y-direction motor 10a and the Z-direction motor 12a in association with one or a plurality of the size (the length, the product number or the like) of the pipette tip 18 attached to the sucker 16, the type (the product number or the like) of the microscope to which the cell picking device 2 is attached and the type (the product number or the like) of the container 28 installed on the sample mounting stage 24. Then, the cell picking device 2 can be applied to various types of the pipette tips 18, the microscopes and the containers 28. That is, because the height of the bottom portion of the container varies depending on the type of the container 28, if the motor drive amount is to be the same regardless of the type of the container 28, the arrival position of the tip of the pipette tip 18 varies depending on the type of the container 28 to be used. As such, the motor drive amount is stored for each type of the container 28, the information in regard to the container 28 to be used is input by a user operation, and the motor drive amount corresponding to the input information of the container 28 is automatically read and set. Thus, the arrival position of the tip of the pipette tip 18 can be the same. The same applies to the size of the pipette tip 18 and the type of the microscope.

When performing cell picking, the controller 20 controls an operation of the driver 6 based on the information stored in the drive amount storage 22 such that the tip of the pipette tip 18 arrives at the predetermined position on the sample mounting stage 24 and then moves in the horizontal direction to desorb and suck the cell adsorbed on the bottom surface of the container 28. When this cell picking operation is performed, the user adjusts the position of the container 28 in advance using the microscope such that the cell to be picked is present in the predetermined position.

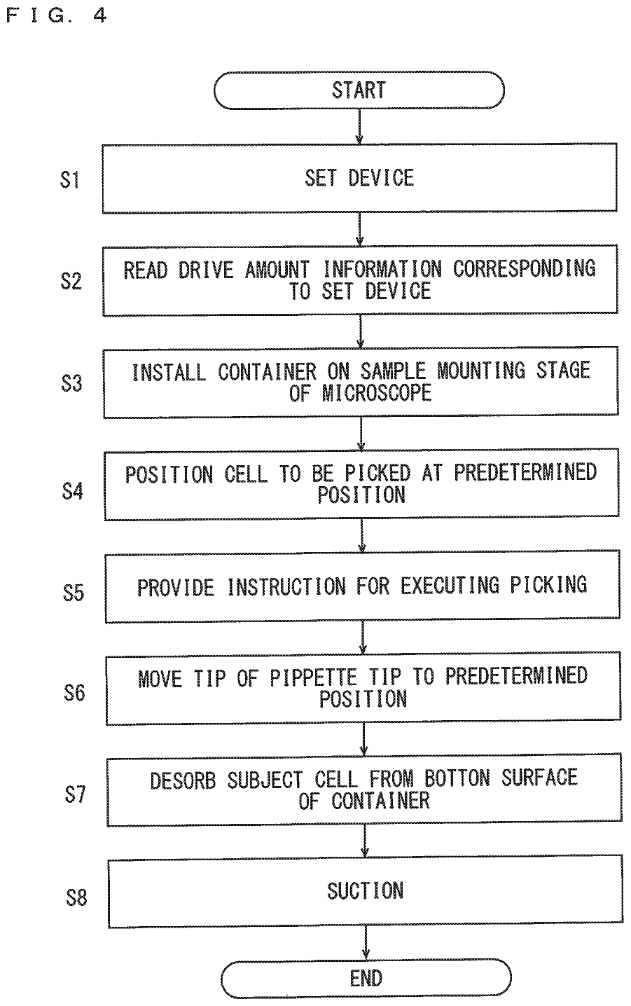

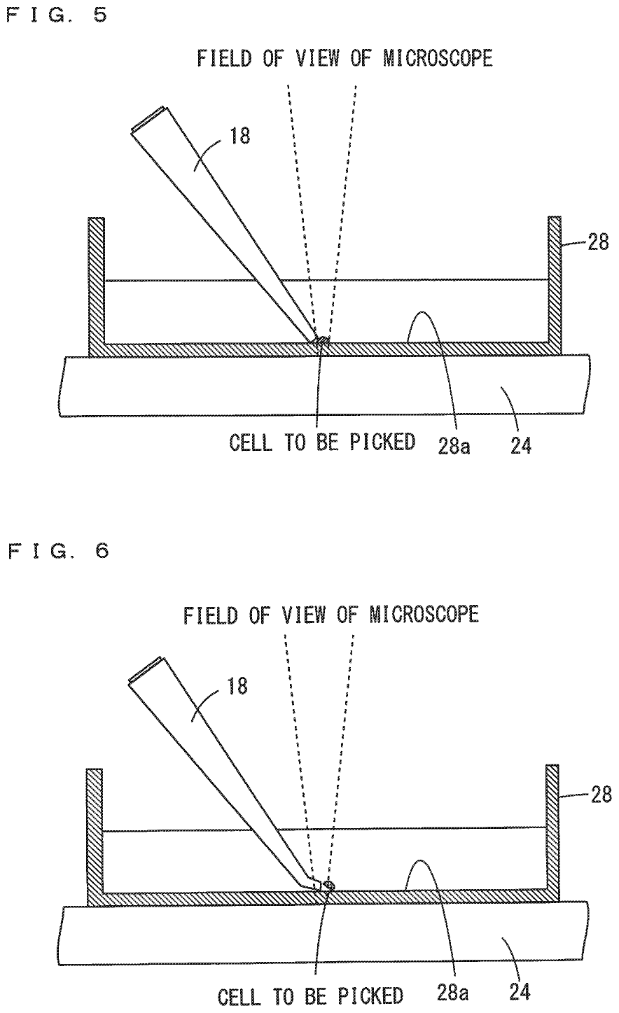

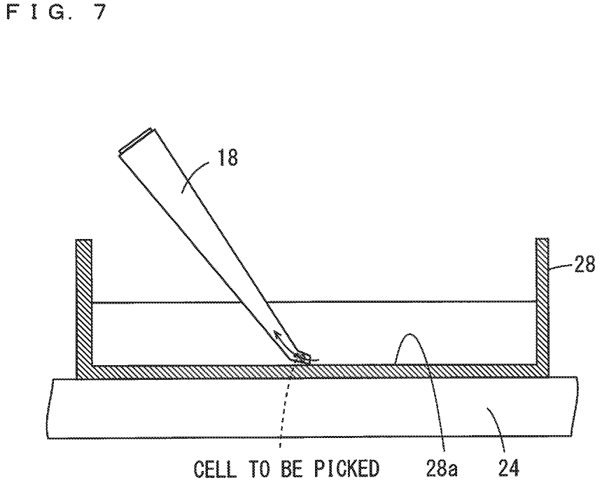

One example of the cell picking work with use of the cell picking device 2 will be described in the flow chart of FIG. 4 with reference to FIGS. 5 to 7.

First, the user sets the information in regard to the devices used for cell picking such as the pipette tip 18, the container 28 and the microscope in the device (step S1). The controller 20 reads the information in regard to the drive amounts of the X-direction motor 8a, the Y-direction motor 10a and the Z-direction motor 12a corresponding to the device that is set based on the input made by the user from the drive amount storage 22 (step S2).

The user installs the container 28 on the sample mounting stage 24 of the microscope (step S3), and adjusts the position of the container 28 such that the cell to be picked is in the field of view of the microscope (step S4). After the position adjustment of the cell to be picked is finished, the user inputs an instruction for executing the cell picking to the controller 20 (step S5).

When the cell picking is executed, the controller 20 controls an operation of the driver 6 such that the tip of the pipette tip 18 is arranged at the predetermined position, that is, the position at which the pipette tip 18 can scrape off the cell arranged in the position in the field of view of the microscope as shown in FIG. 5 (step S6).

The controller 20 slightly moves the sucker 16 toward the tip of the pipette tip 18 with the tip of the pipette tip 18 in contact with a bottom surface 28a of the container 28. Thus, the tip of the pipette tip 18 is pressed obliquely against the bottom surface 28a of the container 28, whereby the tip of the pipette tip 18 is elastically bent, and the tip of the pipette tip 18 is moved in the horizontal direction along the bottom surface 28a of the container 28. By this operation, the cell to be picked, which has been adsorbed on the bottom surface 28a of the container 28, is desorbed from the bottom surface 28a (step S7). Thereafter, the cell to be picked that have been desorbed from the bottom surface 28a of the container 28 is sucked from the tip of the pipette tip 18 (step S8).

While the tip of the pipette tip 18 is pressed against the bottom surface 28a of the container 28 such that the pipette tip 18 is elastically deformed and the cell adsorbed on the bottom surface 28a of the container 28 is scrapped off from the bottom surface 28a in the above-mentioned cell picking operation, the present invention is not limited to this. The pipette tip 18 may be moved in the horizontal direction with the tip of the pipette tip 18 in contact with the bottom surface 28a of the container 28 such that the cell adsorbed on the bottom surface 28a of the container 28 is scrapped off from the bottom surface 28a.

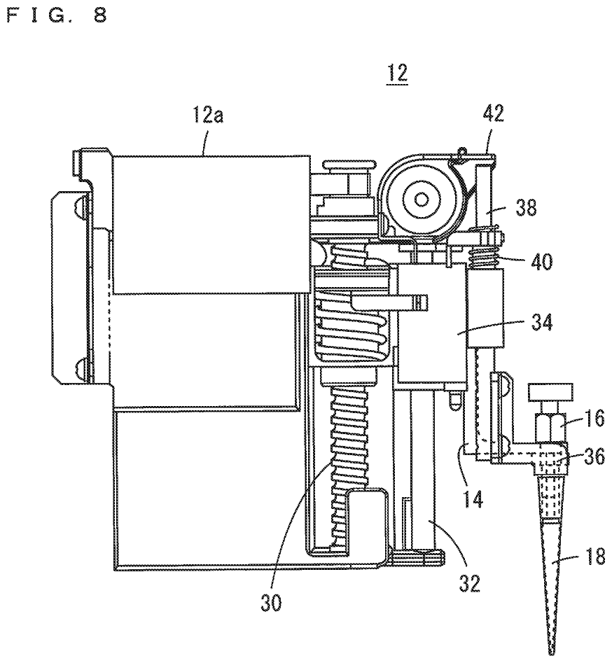

FIG. 8 shows one example of the specific configuration of the sucker drive mechanism 12 of the driver 6.

The sucker drive mechanism 12 includes a ball screw 30 rotated by the Z-direction motor 12a, and a mobile object 34 that moves along a guide rail 32 in the axial direction of the ball screw 30 as the ball screw 30 is rotated. The holding arm 14 holding the sucker 16 moves in connection with the mobile object 34, and the sucker 16 moves in the axial direction of the pipette tip 18 as the ball screw 30 is rotated.

The sucker drive mechanism 12 of this example includes a tip detachment mechanism for automatically detaching the pipette tip 18 from the sucker 16. The sucker detachment mechanism is constituted by a detachment bar 38 attached to the mobile object 34 via a coil spring 40, a detachment arm 36 attached to one end of the detachment bar 38 and a stopper 42 provided at the other end of the detachment bar 38.

The detachment bar 38 is provided in parallel to the ball screw 30. The detachment arm 36 slidably holds a portion of the body of the sucker 16 that is located at a position closer to the proximal end than to the pipette tip 18. The stopper 42 is provided at a position where abutting against the other end of the detachment bar 38 when the mobile object 34 arrives at a certain position (a position that is spaced apart upwardly from a default position by 5 mm in the diagram, for example).

With the above configuration, in a case where the mobile object 34 moves further from the position where the other end of the detachment bar 38 abuts against the stopper 42 toward the other end of the detachment bar 38 (the upper field of the drawing), the detachment bar 38 stops moving. Meanwhile, the holding arm 14 moves toward the pivotal end of the pipette tip 18 together with the mobile object 34. Thus, the detachment arm 36 moves toward the tip of the pipette tip 18 relative to the sucker 16, and the detachment arm 36 presses the pivotal end of the pipette tip 18 toward the tip. Thus, the pipette tip 18 is detached from the sucker 16.

In a case where the driver 6 includes the above-mentioned tip detachment mechanism, the controller 20 is preferably configured to move the pipette tip 18 to a predetermined disposal site after a cell picking operation is completed or at a point in time instructed by the user, and detach the pipette tip 18 from the sucker 16 at the disposal site. The tip detachment mechanism is not an essential configuration in the present invention.

### REFERENCE SIGNS LIST

2 Cell Picking Device
4 Vertical Axis
6 Driver
8 Horizontal Rotator
8a X-direction Motor
10 Vertical Rotator
10a Y-direction Motor
12 Sucker Drive Mechanism
12a Z-direction Motor
14 Holding Arm
16 Sucker
16a Suction Driver
18 Pipette Tip
20 Controller
22 Driver Amount Storage
24 Sample Mounting Stage (Microscope)
26 Lens (Microscope)
28 Container
28a Bottom Surface of Container
30 Ball Screw
32 Guide Rail
34 Mobile Object
36 Detachment Arm
38 Detachment Bar
40 Coil Spring
42 Stopper

1. A cell picking device that is installed around a microscope and is used to pick a cell in a container installed on a sample mounting stage of the microscope, comprising:
a sucker having a pipette tip attached to a tip portion of the sucker and is used to suck the cell from a tip of the pipette tip;
a driver configured to cause the sucker to perform a suction operation and move the sucker in a horizontal-plane direction and an axial direction of the pipette tip with the sucker inclined with respect to a vertical direction; and
a controller that controls the driver, and is configured to move the sucker such that the tip of the pipette tip moves in a horizontal direction while being in contact with a bottom surface of the container installed on the sample mounting stage in a predetermined position on the sample mounting stage to desorb a cell arranged in the predetermined position from the bottom surface of the container, and suck the cell desorbed from the bottom surface of the container from the tip of the pipette tip.

2. The cell picking device according to claim 1, wherein
the controller is configured not to carry out suction by the sucker until the cell is desorbed from the bottom surface of the container, and suck the cell from the tip of the pipette tip after the cell is desorbed from the bottom surface of the container.

3. The cell picking device according to claim 1, wherein
the controller is configured to move the tip of the pipette tip with the tip being in contact with the bottom surface of the container by obliquely pressing the tip of the pipette tip against the bottom surface of the container and elastically bending the tip of the pipette tip.

4. The cell picking device according to claim 1, wherein
the driver includes a sucker angle adjustment mechanism for adjusting an inclination angle of the sucker with respect to the vertical direction.

5. The cell picking device according to claim 4, wherein
the sucker angle adjustment mechanism is configured to rotate the sucker in a vertical plane by a motor.

6. The cell picking device according to claim 1, comprising a drive amount storage that stores information in regard to a drive amount of the driver required to position the tip of the pipette tip at the predetermined position, wherein
the controller is configured to control the driver using the information stored in the drive amount storage when the cell is picked from the container installed on the sample mounting stage of the microscope.

7. The cell picking device according to claim 6, wherein
the drive amount storage stores information relating to the drive amount for each of a size of the pipette tip attached to the tip portion of the sucker, a type of the microscope and/or a type of the container installed on the sample mounting stage of the microscope, and
the controller is configured to determine a drive amount of the driver required to position the tip of the pipette tip at the predetermined position based on the information relating to the drive amount stored in the drive amount storage, and the size of the pipette tip, the type of the microscope and/or the type of the container that are set based on an input by a user, and control the driver using the determined drive amount.

8. The cell picking device according to claim 1, wherein
the driver includes a tip detachment mechanism configured to detach the pipette tip from the sucker by utilizing an operation of moving the sucker in the axial direction of the pipette tip.
